(19) United States
(12) Patent Application Publication
(10) Pub. No.: US 2008/0085315 A1
(43) Pub. Date: Apr. 10, 2008

(54) Amorphous ezetimibe and the production thereof

(76) Inventors: John Alfred Doney, Washington, DC (US); Albert Walter Brzeczko, Baltimore, MD (US); Christopher Steven Shores, Marriottsville, MD (US)

Correspondence Address: INTERNATIONAL SPECIALTY PRODUCTS;Attn: William J. Davis, Esq.,, Legal Department, Building No. 8-2, 1361 Alps Road, Wayne, NJ, 07470, US

(21) Appl. No.: 11/545,350

(22) Filed: Oct. 10, 2006

(51) Int. Cl. A61K 31/397; A61K 9/14

(52) U.S. Cl. 424/486; 424/488; 514/210.02

(57) ABSTRACT

Ezetimibe compositions of enhanced bioavailability are described that contain ezetimibe with at least one solubility-enhancing polymer. Described methods to produce the bioenhanced products comprise solvent spray drying. One aspect of the method includes the steps of providing a mixture comprising ezetimibe, a solubility-enhancing polymer and a single solvent, a solvent blend or solvent/non-solvent blend and then evaporating the mixture to form amorphous ezetimibe.

### BACKGROUND OF THE INVENTION

The present invention is directed to compositions containing amorphous ezetimibe and methods for producing amorphous ezetimibe. More particularly, the present invention relates to compositions and methods for preparing amorphous ezetimibe utilizing at least one solubility-enhancing polymer. In accordance with one embodiment, the ezetimibe is dissolved in a solvent containing the polymer. In yet another embodiment, a blend of solvent/non-solvent for the polymer is employed. The amorphous ezetimibe product can be produced by any method suitable to the composition. When necessary, solvent can be removed from compositions to yield the amorphous ezetimibe product. In one further development of the invention, ezetimibe-polymer-solvent (or a solvent/non-solvent blend) is spray dried to produce ezetimibe in a form that exhibits improved bioavailability. The bioenhanced ezetimibe composition can be prepared by methods other than spray drying as recognized by those skilled in the art. Those methods include, without limitation: melt extrusion, spray congealing, granulation and freeze drying. In accordance with particular embodiments of the invention, a significant portion of the ezetimibe is provided in the amorphous state. In accordance with certain embodiments, the ezetimibe is converted almost entirely to the amorphous state. In one preferred embodiment of the invention, the ezetimibe is converted to the completely amorphous state.

Ezetimibe (EZE) is known by the chemical name 1-(4-fluorophenyl)-3(R)-[3-(4-fluorophenyl)-3(S)-hydroxypropyl]-4(S)-(4-hydroxyphenyl)-2-azetidinone. The empirical formula of ezetimibe is C₂₄H₂₁F₂NO₃ and its molecular weight is 409.4. Ezetimibe is a lipid-lowering chemical that inhibits the absorption of dietary cholesterol and related compounds. Ezetimibe is a white, crystalline powder that is insoluble in water and exhibits a range of solubilities in organic solvents. The melting point of pure EZE is about 163° C.

EZE is available commercially in two products. Zetia®, marketed by Merck/Schering-Plough Pharmaceuticals, is a tablet containing 10 mg of EZE. It is formulated with croscarmellose sodium, lactose monohydrate, magnesium stearate, microcrystalline cellulose, povidone, and sodium lauryl sulfate. Vytorin®, also marketed by Merck/Schering-Plough Pharmaceuticals, contains 10 mg EZE along with 10, 20, 40, or 80 mg of simvastatin.

It is desirable to provide methods of producing ezetimibe exhibiting enhanced bioavailability compared to the crystalline form of the compound. By converting a substantial portion of crystalline ezetimibe to the amorphous state, the aqueous solubility and bioavailability are increased. Furthermore, ezetimibe presented as an amorphous solid may facilitate manufacturing of both the active ingredient and the finished product and enable the use of reduced size dosage forms. Moreover, the selective customization of the properties of particles comprising ezetimibe can offer intriguing opportunities for pharmaceutical production and drug delivery. The morphology of individual particles plays a central role in this pursuit, since morphology directly influences bulk powder properties, such as density, residual solvent content, and flowability. In addition, techniques that modify particle shape and interior structure may profoundly affect pharmacokinetic properties, such as drug release rate, solubility, and bioavailability. Thus, the ability to design particle morphology has significant implications for the production process and product attributes.

### SUMMARY OF THE INVENTION

The present invention provides methods for producing amorphous ezetimibe and compositions containing amorphous ezetimibe. A composition comprising a solid dispersion of ezetimibe and at least one solubility-enhancing polymer wherein the ezetimibe in the dispersion is substantially amorphous is provided in certain embodiments of the present invention. In one aspect, the disclosed invention describes the conversion of crystalline ezetimibe to the amorphous state. One method for producing this conversion is through solvent spray drying. Other techniques that accomplish this conversion include, without limitation: flash solvent evaporation, melt-congeal spraying, freeze drying, and melt-extrusion. These methods can use a single solubility-enhancing polymer or blends of polymers. The degree of ezetimibe amorphous conversion depends on various factors, including, but not limited to, polymer type and amount and processing conditions. In accordance with certain aspects of the invention, a single organic solvent, blends of solvents, or solvent/non-solvent blends can be used.

In one aspect, the invention relates to spray-dried powders or granulated products comprising amorphous ezetimibe. In addition, the resulting powders produced in accordance with certain embodiments typically possess lower residual solvent content and higher tap density than their counterparts produced by conventional methods, due to a change in the particle morphology and size. When applied to produce pharmaceutical products, a system of polymers can be used to modify not only particle morphology, but also the pharmacokinetic properties of the active.

One aspect of the invention involves amorphous ezetimibe prepared from compositions containing ezetimibe and a solubility-enhancing polymer in a solvent or solvent blend. This solvent or solvent blend includes one or more solvents in which the polymer is soluble. The term “soluble” means that the attractive force between polymer and solvent molecules is greater than the competing inter- and intramolecular attractive forces between polymer molecules. For simplicity, this solvent is simply called “solvent.” Compositions also are described in which the solvent blend contains a solvent for which the opposite is true: The attractive force between polymer and solvent molecules is less than the inter- and intramolecular attractive force between polymer molecules. This second solvent is termed the “non-solvent.” The polymer may swell but does not dissolve in the non-solvent. In accordance with one embodiment of the invention, a solubility-enhancing polymer and a suitable solvent/non-solvent blend are provided. Additionally, the solvent possesses a lower boiling point than the non-solvent. Preferably, the solvent and non-solvent are miscible. The ratio of solvent to non-solvent is such that the polymer can be considered “dissolved” in the solvent system.

Unique particle properties can be created by evaporating the solvent/non-solvent blend. For example, this evaporation can occur during the spray drying of the feed solution or granulation processes. Atomized droplets containing a blend of solvents will experience a change in the total solvent composition due to evaporation. The method appears to be independent of how the droplets are generated or atomized. Initially, the polymer exists in a dissolved state, due to a sufficient amount of the solvent. As it evaporates (the solvent boils at a lower temperature than the non-solvent), the concentration of non-solvent in the droplet increases. Eventually, the solvent composition is insufficient to maintain the polymer in solution. In doing so, the polymer collapses from solution. This change in polymer conformation can alter the evaporation dynamics of the droplet to create particle morphologies that influence final powder properties.

The use of a solvent/non-solvent blend system has been found to provide additional benefits beyond the benefits obtained with a solvent only system. This solvent/non-solvent approach can produce a spray dried powder of lower residual solvent content and smaller particle size. A further consequence of this engineered particle morphology is the increase in bulk powder density. Increased powder density is an important attribute for many applications. The extent of polymer collapse—and therefore the net effect on the spray dried powder properties—depends on the polymer solvation factors, such as the initial ratio of solvent to non-solvent, the polymer chemical structure and the polymer molecular weight. In addition to reducing residual solvent content and increasing density, the primary polymer may be paired with the solvent/non-solvent system in order to affect not only the morphology of the particle, but also that of the ezetimibe, and thereby affect the ezetimibe loading, crystallinity, solubility, stability and release.

The presence of additional polymers may contribute to the final particle morphology by their interaction with the first polymer and the solvent system. These additional polymers may also be advantageous to create special release properties of the active. For example, the primary polymer may be paired with the solvent/non-solvent system in order to affect particle morphology, and thereby residual solvent content and bulk powder density. Additional polymeric adjuvants may be added to serve additional purposes: further inhibit active recrystallization, further maximize active concentration, and further enhance/delay/retard dissolution rate. To accomplish these functionalities, it is necessary to suitably match the adjuvant solubilities with the solvent blend selected for the primary polymer.

### BRIEF DESCRIPTION OF THE DRAWINGS

FIG. 1 is a plot of heat flow vs. temperature for compositions produced in accordance with Example 1.

FIG. 2 is a plot of heat flow vs. temperature for compositions produced in accordance with Example 2.

FIG. 3 is a plot of EZE percent release as a function of time for compositions produced in accordance with Example 2.

FIG. 4A is a plot of EZE plasma concentration as a function of time for male Sprague Dawley rats in accordance with Example 3.

FIG. 4B is a plot of EZE-G plasma concentration as a function of time for male Sprague Dawley rats in accordance with Example 3.

FIG. 5 is a plot of heat flow as a function of temperature for the composition produced in accordance with in Example 4.

FIG. 6A is a photomicrograph of the composition produced in accordance with Example 2.

FIG. 6B is a photomicrograph of the composition produced in accordance with Example 4.

FIG. 7 is a plot of EZE percent release as a function of time for a composition produced in accordance with Example 4.

### DETAILED DESCRIPTION OF THE INVENTION

The term “comprising” encompasses the more restrictive terms “consisting essentially of” and “consisting of.”

All percentages, ratios and proportions used herein are by weight unless otherwise specified.

The term “active metabolite” refers to products of in vivo modification of ezetimibe which have therapeutic or prophylactic effect. Ezetimibe glucuronide is the primary active metabolite of ezetimibe.

Bioavailability refers to the degree to which the active or active metabolite becomes available in the body after administration. Typically, plasma samples are taken and analyzed for the plasma concentration of the parent compound and/or its active metabolite. These data may be expressed as Cₘₐₓ, the maximum amount of active ingredient found in the plasma, or as AUC, the area under the plasma concentration time curve. Enhanced bioavailability may be evidenced by an increase in Cₘₐₓ and/or AUC for the active, the active metabolite or both. Compositions in accordance with certain aspects of the invention exhibit enhanced bioavailability compared to a control composition.

The term “solid dispersion” as used herein refers to a system in a solid state comprising at least two components, wherein one component is dispersed evenly throughout the other component or components. The term “solid dispersion” includes systems having small particles either completely crystalline, completely amorphous or any state in between, typically less than about 1 μm in diameter, of one phase dispersed in another phase.

The term “solid solution” as used herein refers to a type of solid dispersion wherein one component is molecularly dispersed throughout another component such that the system is chemically and physically uniform and homogeneous throughout. These systems do not contain any significant amounts of active ingredients in their crystalline or microcrystalline state as evidenced by thermal analysis or X-ray diffraction.

The term “solubility-enhancing polymer” refers to a polymer that provides at least one of the following properties as a result of its presence in the composition compared to a control composition without the solubility-enhancing polymer:

a) an increase in initial release of at least about 25%

b) an increase in extent of release of at least about 25%

c) an increase in maximum plasma concentration of at least about 25%

d) an increase in AUC₀₋₂₄ₕ of at least about 25%.

Although the following description is primarily directed to the preparation of a spray-dried composition containing ezetimibe, the present invention is not limited to ezetimibe spray-dried compositions. The scope of the invention includes other methods described herein that are also useful in converting ezetimibe to the amorphous state and corresponding enhanced bioavailability. Those methods include, without limitation: melt extrusion, spray congealing, granulation and freeze drying.

There is no condition placed on the state of the compositions other than amorphous ezetimibe combined with one or more solubility-enhancing polymer(s). The term “combined” includes, but is not limited to: blended, co-mingled, dissolved, extruded, granulated, melted, milled, mixed, sieved, slurried, sprayed, stirred, and the combination of these and other methods. Other techniques may be identified by those skilled in the art.

In accordance with one embodiment, the present invention is related to a method for preparing a spray-dried composition by providing a mixture containing ezetimibe and a polymer in a single solvent, a solvent blend or a blend of a solvent and a non-solvent for the polymer and spray drying the mixture to form the spray-dried composition.

In accordance with one aspect of the invention, a polymer system is provided comprising a polymer—called the primary polymer—and a suitable solvent or solvent blend. This approach comprises a solvent in which the polymer is soluble. Guidance in defining polymer solubility is provided by the expansion coefficient (α):

$$α = \frac{{( {\overline{r}}^{2} )}^{1 / 2}}{{( {\overline{r}}_{o}^{2} )}^{1 / 2}} \qquad ( §1 )$$

where r̅² is the mean-square distance between chain ends, and r̅ₒ̅² is the unperturbed dimension. (Equation § 1 can be written for branched polymers in an analogous manner, using square-average radius of gyration about the center of gravity, s̅², and the corresponding unperturbed dimension, s̅ₒ̅².) Polymer solubility is provided when α is unity or greater, and solvents that satisfy this condition are called “good solvents,” or simply “solvents.” Solvents uncoil (or expand) the polymer molecule, since the polymer-solvent attractive force is greater than that of polymer-polymer. Light scattering methods, such light scattering detectors (e.g., Triple Detector Array, Viscotek Corp.), can be used to determine the variables expressed in equation § 1. These concepts are defined in the text Polymer Chemistry, An Introduction, by Malcolm P. Stevens, which is incorporated by reference.

When α equals unity, a special condition exists in that polymer-solvent and polymer-polymer forces are balanced. Solvents that enable this condition are called θ solvents. Within the context of this invention, solvents are considered “good solvents” when α is about equal to 1 or more. It is appreciated that temperature influences α, such that a good solvent may be transformed into a non-solvent merely by changing the temperature.

In yet another embodiment of this invention, the solvent blend also contains a solvent for which the opposite is true: Polymer-polymer forces dominate polymer-solvent forces. In this case, α is less than one and the solvent is termed a “non-solvent,” because the polymer exists in a collapsed state. In accordance with one embodiment of the invention, one polymer is provided with a suitable solvent/non-solvent blend. The blend of solvent/non-solvent maintains a solvated state of the polymer, such that the polymer can be considered “dissolved” in the solvent system. Additionally, the solvent possesses a lower boiling point than the non-solvent. (Solvent/non-solvent pairs that form an azeotrope do not satisfy this criterion.) Preferably, the solvent and non-solvent are miscible.

Unique particle properties can be created by evaporating the solvent/non-solvent blend. For example, this evaporation can occur during the spray drying of the feed solution or granulation processes. Atomized droplets containing a blend of solvents will experience a change in the total solvent composition due to evaporation. The method appears to be independent of how the droplets are generated or atomized. Initially, the polymer exists in a dissolved state, due to a sufficient amount of the solvent. As it evaporates (the solvent boils at a lower temperature than the non-solvent), the concentration of non-solvent in the droplet increases. Eventually, the solvent composition is insufficient to maintain the polymer in solution. In doing so, the polymer collapses and precipitates from solution. This change in polymer conformation can alter the evaporation dynamics of the droplet to create particle morphologies that influence final powder properties. Examples of suitable polymer/solvent/non-solvent combinations include, without limitation, polyvinylpyrrolidone/dichloromethane/acetone, polyvinylpyrrolidone-co-vinyl acetate/acetone/hexane, and ethylcellulose/acetone/water.

Unique particle architectures are created by precipitation of the polymer when the non-solvent concentration exceeds a critical value. This critical ratio R_{c} can be defined:

$${R}_{c} = \frac{mass nonsolvent}{mass solvent + nonsolvent} ,$$

which is the maximum fraction of the non-solvent before polymer collapse occurs. The ratio R_{c} for a given system can be determined experimentally by identifying the mass fractions of each component that produce a significant increase in solution turbidity. If an R_{c} value can be identified for a system, then the system comprises a solvent/non-solvent blend. One example is a solution consisting of about 10% (w/w) polyvinylpyrrolidone, 18% (w/w) dichloromethane, and 72% (w/w) acetone, for which R_{c} equals 0.80. Polymer systems will typically be used at solvent/non-solvent blends that are below the R_{c} value for the system. It may be advantageous to formulate more complex polymer/solvent systems in order to control particle morphology/size as well as the crystallinity, solubility, bioavailability and/or release characteristics of the ezetimibe.

The present invention in accordance with other embodiments provides a method to increase the density of spray-dried powders. Typically, spray drying produces sphere-like particles with some degree of interior void. This void increases particle bulk without mass and creates low-density material. Adding a non-solvent to the working solution/dispersion changes the particle size and morphology, leading to an increase in density. Particles may be smaller, wrinkled, dimpled, and/or collapsed compared to those prepared using only solvent. The solvent/non-solvent approach also reduces the mean particle size, allowing the powder to pack better. In addition, powder flow and powder-powder mixing properties are enhanced.

The present invention in accordance with certain aspects provides a method to reduce or eliminate the need for secondary drying of spray-dried powders and granulated materials. These products often contain residual solvent, and it is desirable or necessary to produce a drier product. A high residual solvent content can result from formulation or processing limitations. The general practice has been to use a solvent that dissolves the solids being spray dried. In doing so, solvent can be trapped inside the spray dried powder or granulated bead due to case hardening. The intentional pairing of a lower-boiling solvent with a higher-boiling non-solvent for the materials being processed can yield products of lower residual solvent due to the effect(s) of the non-solvent on the process polymers.

The present invention may further provide a method to enhance the aqueous solubility and modify the release of active ingredients through selection of a polymer system with the solvent or solvent/non-solvent blend. The polymer system is chosen so that one (or more) polymer(s) work with the solvent/non-solvents to create novel particle morphologies. Additional polymer(s) may be added as needed to affect the solubility and release properties of the ezetimibe, as well as particle morphology. Enhanced solubility can be achieved by a number of factors, including (but not limited to): improved wettability, creation of amorphous drug forms, stabilization against recrystallization, and/or co-solvation effects. In doing so, a supersaturatured solution of the ezetimibe is produced. “Modified release” refers to changing the time frame in which the active is released, i.e., immediate, delay, extended. These modified releases are created by matching functional polymer(s) with the appropriate solvent/non-solvent blend.

Solvents and non-solvents suitable for use in the process of the present invention can be any organic compound (including water) in which the primary polymer is soluble in the case of solvents, or insoluble, in the case of non-solvents. The choice and ratio of solvent/non-solvent depends on the choice of the primary polymer. Accordingly, the identification of an organic compound as a solvent or non-solvent depends on the primary polymer. Therefore, a solvent in one system may be a non-solvent in another. Particularly useful solvents and non-solvents include, but are not limited to: acetic acid, acetone, acetonitrile, anisole, 1-butanol, 2-butanol, butyl acetate, tert-butylmethyl ether, chlorobenzene, chloroform, cumene, cyclohexane, 1-2-dichloroethane, dichloromethane, 1-2-dimethoxyethane, N—N-dimethylacetamide, N—N-dimethylformamide, 1-4-dioxane, ethanol, 2-ethoxyethanol, ethyl acetate, ethylene glycol, ethyl ether, ethyl formate, formamide, formic acid, heptane, hexane, isobutyl acetate, isopropyl acetate, methanol, methyl acetate, 2-methoxyethanol, 3-methyl-1-butanol, methylbutylketone, methylcyclohexane, methylethyl ketone, methylisobutyl ketone, 2-methyl-1-propanol, N-methylpyrollidone, nitromethane, pentane, 1-pentanol, 1-propanol, 2-propanol, propyl acetate, pyridine, sulfolane, tetrahydrofuran, tetralin, 1-2-2-trichloroethene, toluene, water, and xylene. Mixtures of solvents and mixtures of non-solvents can also be used. In accordance with particular embodiments, solvent blends at the azeotropic composition (which boil at one common temperature) can comprise either the solvent or non-solvent, but not the solvent/non-solvent blend.

Solubility-enhancing polymers that are suitable for use in the mixtures of the present invention should result in conversion of at least some of the crystalline ezetimibe to the amorphous state. In accordance with those embodiments wherein a solvent/non-solvent blend is used, at least one polymer should be soluble in the solvent and not soluble in the non-solvent. Specific examples of useful polymers include, but are not limited to: aliphatic polyesters (e.g., poly D-lactide), carbohydrates (e.g., sucrose), carboxyalkylcelluloses (e.g., carboxymethylcellulose), alkylcelluloses (e.g., ethylcellulose), gelatins, hydroxyalkylcelluloses (e.g., hydroxymethylcellulose), hydroxyalkylalkylcelluloses (e.g., hydroxyethylmethyl cellulose), hydroxyalkylalkylcellulose derivatives (e.g. hydroxypropylmethyl cellulose phthalate, hydroxypropylmethyl cellulose acetate succinate) polyamines (e.g., chitosan), polyethylene glycols (e.g., PEG 8000, PEG 20000), methacrylic acid polymers and copolymers (e.g., Eudragit® series of polymers by Rohm Pharma, GmbH), homo- and copolymers of N-vinyl pyrrolidone (e.g., polyvinylpyrrolidone, polyvinylpyrrolidone-co-vinyl acetate), homo- and copolymers of vinyllactam, polysaccharides (e.g., alginic acid), poly glycols (e.g., propylene glycol, polyethylene glycol), polyvinyl esters (e.g., polyvinyl acetate), and refined/modified shellac. The amount of the polymer present in the mixture may range from about 1% to about 95%, more particularly from about 5% to 90%, by weight of the mixture, and in accordance with certain embodiments from about 25% to 75% by weight. Blends of polymers may also be used.

The spray-dried mixture includes ezetimibe as an active ingredient. The mixture may contain from about 1% to about 95% active, more particularly from about 20% to about 80% active, depending on the desired dose of the active. The weight ratio of ezetimibe to polymer typically will be from about 95% ezetimibe:5% total polymer to about 5% ezetimibe:95% total polymer, more particularly from about 70% ezetimibe:30% total polymer to about 30% ezetimibe:70% total polymer and in accordance with certain aspects from about 60% ezetimibe:40% total polymer to about 40% ezetimibe:60% total polymer.

In accordance with certain aspects of the present invention, compositions are provided wherein at least a portion of ezetimibe is in the amorphous state. The term “amorphous” refers to a compound in a non-crystalline state. In other words, an amorphous compound lacks long-ranged, defined crystalline structure. In accordance with certain embodiments of the present invention, at least some, more particularly at least about 10%, at least about 25%, or at least about 40% of the ezetimibe in the composition is in an amorphous form. In other embodiments, at least a major portion of the compound in the composition is amorphous. As used herein, the term “a major portion” of the compound means that at least about 50% of the compound in the composition is in the amorphous form, rather than the crystalline form. More particularly, the compound in the composition may be substantially amorphous. As used herein, “substantially amorphous” means that the amount of the compound in the crystalline form does not exceed about 25% (i.e., more than about 75% of the compound is in the amorphous form). In accordance with particular embodiments of the invention, the compound in the composition is “almost completely amorphous” meaning that the amount of drug in the crystalline form does not exceed about 10% (i.e., more than about 90% of the compound is in the amorphous form). Compositions are also provided wherein the compound in the composition is considered to be “completely amorphous” meaning that the crystalline form of the drug does not exceed about 1%.

Amorphous materials lack some measurable properties, such as melting endotherms as measured by differential scanning calorimetry that characterize crystalline forms. Amounts of crystalline drug may be measured by powder x-ray diffraction (PXRD), differential scanning calorimetry (DSC), or any other standard quantitative analysis. The amounts of crystalline ezetimibe present in the composition may be detected by other standard measurement known to those of ordinary skill in the art. It is appreciated that the measurement of such properties may be dependent on instrument type and sensitivity.

The spray dried product produced in accordance with certain aspects of the present invention provides enhanced bioavailability of ezetimibe compared to products containing the principle crystalline form. The increased bioavailability of the active can also lead to reduced dosage sizes and dose amounts for the active. Applicants have also determined that the rate of drug release can be controlled through proper selection of the polymers added into the solvent solution for processing. In certain embodiments of the invention, the process is spray drying.

The spray dried mixture may also contain additional polymeric materials that can modify properties of the composition. For example, certain polymers can be included to control particle morphology/size as well as the bioavailability and release characteristics of the active ingredient. Additional polymers may also be included in the mixture to further inhibit active recrystallization, further maximize active concentration and further enhance/delay/retard dissolution rate. Additional polymers that can be incorporated into this system are not particularly limited.

The mixture to be spray dried typically contains from about 40% to 99.9% by weight total solvent or solvent/non-solvent, more particularly from about 80% to 95% by weight total solvent or solvent/non-solvent based on the total weight of the mixture. When a solvent/non-solvent blend is used, the critical ratio R_{c} can vary from about 0.01-0.99, more particularly from about 0.1-0.9, still more particularly from about 0.3-0.8.

In addition to the solvent, polymer and ezetimibe, the mixture to be spray dried may also include other ingredients to improve performance, handling or processing of the mixture. Alternatively, these ingredients also may be admixed into the already-prepared ezetimibe-polymer by methods including, but not limited to tumble blending and granulation technologies. Typical ingredients include, but are not limited to, surfactants, pH modifiers, fillers, complexing agents, solubilizer, pigments, lubricants, glidants, flavor agents, plasticizers, taste masking agents, etc., which may be used for customary purposes and in typical amounts.

The spray drying apparatus used in the process of the present invention can be any of the various commercially available apparatus. Examples of specific spray drying devices include spray dryers manufactured by Niro Inc. (e.g., SD-Micro®, PSD®-1, PSD®-2, etc.), the Mini Spray Dryer (Buchi Labortechnik AG), spray dryers manufactured by Spray Drying Systems, Inc. (e.g., models 30, 48, 72), and SSP Pvt. Ltd.

Spray drying processes and spray drying equipment are described generally in Perry's Chemical Engineers' Handbook, Sixth Edition (R. H. Perry, D. W. Green, J. O. Maloney, eds.) McGraw-Hill Book Co. 1984, pages 20-54 to 20-57. More details on spray drying processes and equipment are reviewed by Marshall “Atomization and Spray Drying,” 50 Chem. Eng. Prog. Monogr. Series 2 (1954). The contents of these references are hereby incorporated by reference.

The term “spray drying” is used conventionally and, in general, refers to processes involving breaking up liquid mixtures into small droplets and rapidly removing solvent from the mixture in a container (spray drying apparatus) where there is a strong driving force for evaporation of solvent from the droplets. Atomization techniques include two-fluid and pressure nozzles, and rotary atomizers. The strong driving force for solvent evaporation is generally provided by maintaining the partial pressure of solvent in the spray drying apparatus well below the vapor pressure of the solvent at the temperatures of the drying droplets. This may be accomplished by either (1) maintaining the pressure in the spray drying apparatus at a partial vacuum; (2) mixing the liquid droplets with a warm drying gas; or (3) both.

Generally, the temperature and flow rate of the drying gas and spray dryer design are chosen so that the polymer/active solution droplets are dry enough by the time they reach the wall of the apparatus that they are essentially solid and so that they form a fine powder and do riot: stick to the apparatus wall. It is also possible to operate a spray dryer so that product collects on the apparatus wall, and then is collected by removing the material manually, pneumatically, mechanically or other means. The actual length of time to achieve the preferred level of dryness depends on the size of the droplets, the formulation, and spray dryer operation. Following the solidification, the solid powder may stay in the spray drying chamber for 5-60 seconds, further evaporating solvent from the solid powder. The final solvent content of the solid dispersion as it exits the dryer should be low, since this improves the stability of the product. Generally, the residual solvent content of the spray-dried composition should be less than about 10% by weight and preferably less than about 2% by weight. In accordance with certain embodiments, the residual solvent content is within the limits set forth in the International Conference on Harmonization (ICH) Guidelines. Although not typically required in accordance with certain aspects of the present invention, because the presence of a non-solvent produces a spray-dried powder of lower residual solvent content, it may be useful in accordance with certain embodiments of the present invention to subject the spray-dried composition to further drying to lower the residual solvent to even lower levels. Methods to further lower solvent levels include, but are not limited to fluid bed drying, infra-red drying, tumble drying, vacuum drying, and combinations of these and other processes. Additional detail with respect to a particular spray drying process is described in more detail in the examples. However, the operating conditions to spray dry a powder are well known in the art and can be easily adjusted by the skilled artisan. Furthermore, the examples describe results obtained with a laboratory scale spray dryer. One of ordinary skill in the art would readily appreciate the variables that must be modified to obtain similar results with a production scale unit.

As indicated above, the present invention is not limited to amorphous ezetimibe produced by spray drying. In addition to spray drying, compositions of the present invention may be prepared by other processes including, but not limited to, extrusion, spheronization and spray congealing.

Extrusion is a well-known method of applying pressure to a damp or melted composition until it flows through an orifice or a defined opening. The extrudable length varies with the physical characteristics of the material to be extruded, the method of extrusion, and the process of manipulation of the particles after extrusion. Various types of extrusion devices can be employed, such as screw, sieve and basket, roll, and ram extruders.

In melt extrusion, components can be melted and extruded with a continuous process with or without solvent and with or without inclusion of additives. Such a process is well-established and well-known to skilled practitioners in the art.

Spheronization is the process of converting material into spheres, the shape with the lowest surface area to volume ratio. Spheronization typically begins with damp extruded particles. The extruded particles are broken into uniform lengths instantaneously and gradually transformed into spherical shapes. In addition, powdered raw materials, which require addition of either liquid or material from a mixer, can be processed in an air-assisted spheronizer.

Spray congealing is a method that is generally used in changing the structure of the materials, to obtain free flowing powders from liquids and to provide pellets ranging in size from about 0.25 to 2.0 mm. Spray congealing involves allowing a substance of interest to melt, disperse, or dissolve in a hot melt of other additives. The molten mixture is then sprayed into an air chamber wherein the temperature is below the melting point of the formulation components, to provide spherical congealed pellets. The temperature of the cooled air used depends on the freezing point of the product. The particles are held together by solid bonds formed from the congealed melts. Due to the absence of solvent evaporation in most spray congealing processes, the particles are generally non porous and strong, and remain intact upon agitation. The characteristics of the final congealed product depend in part on the properties of the additives used. The rate of feeding and inlet/outlet temperatures are adjusted to ensure congealing of the atomized liquid droplet. The feed should have adequate viscosity to ensure homogeneity. The conversion of molten feed into powder is a single, continuous step. Proper atomization and a controlled cooling rate are critical to obtain high surface area, uniform and homogeneous congealed pellets. Adjustment of these parameters is readily achieved by one skilled in the art.

The spray congealing method is similar to spray drying, except that solvent is not used. Instead, the active ingredient(s) is dispersed and/or melted into a matrix comprising melt-processable polymer(s). Spray congealing is a uniform and rapid process, and is completed before the product comes in contact with any equipment surface. Most actives and additives that are solid at room temperature and melt without decomposition are suitable for this method.

Conventional spray dryers operating with cool inlet air have been used for spray congealing. Several methods of atomization of molten mass can be employed, such as pressure, or pneumatic or centrifugal atomization. For persons skilled in the spray congealing art, it is well known that several formulation aspects, such as matrix materials, viscosity, and processing factors, such as temperature, atomization and cooling rate affect the quality (morphology, particle size distribution, polymorphism and dissolution characteristics) of spray congealed pellets. The spray congealed particles may be used in tablet granulation form, encapsulation form, or can be incorporated into a liquid suspension form.

Ezetimibe compositions produced in accordance with some embodiments of the invention exhibit enhanced bioavailability when present in solid state forms such as solid solutions or solid dispersions. The ezetimibe may be present in such compositions at levels exceeding about 5% by weight, more particularly exceeding about 10%, and in some cases exceeding about 25%, 40%, 50% or even 75% by weight of the composition and still exhibit enhanced bioavailability compared to crystalline forms of the compound.

Certain polymers function as solubility-enhancing polymers in that the presence of the polymer in the composition improves solubility of the ezetimibe under various conditions. The solubility-enhancing polymer provides at least one of the following properties as a result of its presence in the composition compared to a control composition without the solubility-enhancing polymer or to a composition containing the crystalline form of the drug:
a) an increase in initial release of at least about 25%, more particularly at least about 100% and in accordance with certain embodiments at least about 200%
b) an increase in extent of release of at least about 25%, more particularly at least about 100% and in accordance with certain embodiments at least about 200%
c) an increase in maximum plasma concentration of at least about 25%, more particularly at least about 100% and in accordance with certain embodiments at least about 200%
d) an increase in AUC₀₋₂₄ₕ of at least about 25%, more particularly at least about 100% and in accordance with certain embodiments at least about 200%.

Initial release refers to the percent of drug released after 15 minutes in accordance with a standard dissolution test method. Extent of release refers to the percent of drug released after 240 minutes in accordance with the same standard dissolution test method.

In accordance with particular embodiments of the present invention, a composition prepared from a system comprising a polymer and an ezetimibe spray dried from a solvent/non-solvent system as described herein exhibits a dissolution profile wherein the percent active released at some point in time is at least about 25%, more particularly at least about 50% and in certain cases at least about 100% greater than a control composition prepared from a system comprising the same polymer and ezetimibe spray dried from the same solvent without the non-solvent. Preferably these limits are obtained within about 120 minutes, more particularly within about 60 minutes and still more particularly within about 30 minutes. Dissolution profiles can be determined using USP apparatus II (paddles) (VK 7010®, Varian Inc.), with a bath temperature of 37° C. and a paddle speed of 100 rpm for 240 minutes.

In accordance with particular embodiments of the present invention, a composition prepared from a system comprising a polymer and an ezetimibe spray dried from a solvent/non-solvent system as described herein exhibits an increase in bulk density or tap density wherein the density is at least about 25%, more particularly at least about 50% and in certain cases at least about 100% greater than a control composition prepared from a system comprising the same polymer and ezetimibe spray dried from the same solvent without the non-solvent.

Ezetimibe compositions prepared from a solvent/non-solvent system typically result in reduced particle size. In accordance with particular embodiments of the present invention, a composition prepared from a system comprising a polymer and an ezetimibe spray dried from a solvent/non-solvent system as described herein results in a reduction of particle size on the order of at least about 50%, more particularly at least about 100% and in certain cases at least about 300% compared to a control composition prepared from a system comprising the same polymer and ezetimibe spray dried under similar conditions from the same solvent without the non-solvent.

Compositions of the present invention may be delivered by a wide variety of routes, including, but not limited to: buccal, dermal, intravenous, nasal, oral, pulmonary, rectal, subcutaneous, sublingual, and vaginal. Generally, the oral route is preferred.

Compositions of the invention may be presented in a numerous forms. Exemplary presentation forms are powders, granules, and multiparticulates. These forms may be added directly to capsules; compressed to produce tablets, capsules, or pills; or reconstituted by addition of water or other liquids to form a paste, slurry, ointment, suspension or solution. Various additives may be mixed, ground, or granulated with the compositions of this invention to form a material suitable for the above dosage forms.

Compositions of the invention may be formulated in various forms so that they are delivered as a suspension of particles in a liquid vehicle. Such suspensions may be formulated as a liquid or as a paste at the time of manufacture, or they may be formulated as a dry powder with a liquid, typically water, added at a later time but prior to oral administration. Such powders that are constituted into a suspension are often referred to as sachets or oral powders for constitution (OPC). Such dosage forms can be formulated and reconstituted via any known procedure.

Oral, solid-dose pharmaceutical spray dried powders typically have a mean particle size of about 0.5 μm-500 μm and are generally prepared from solutions at concentrations of 1% or more total solids, more particularly from about 2%-50%, and still more particularly from about 3%-30% solids.

Oral, solid dose pharmaceutical granules typically have a mean particle size of about 50 μm-5000 μm. Techniques to produce granules include, but are not limited to, wet granulation and various fluid bed granulating methods.

Pharmaceutical compositions comprising the ezetimibe of enhanced bioavailability described herein may be prepared in accordance with conventional techniques. In accordance with one aspect of the invention, a pharmaceutical dosage form is provided comprising ezetimibe and a disintegrant. The disintegrant used in the composition is preferably of the so-called superdisintegrant type, disintegrants of this type being well-known to the person skilled in the art. As examples of these disintegrants the following can be mentioned: cross-linked polyvinylpyrrolidones, particularly crospovidone, modified starches, particularly sodium starch glycolate, modified celluloses, particularly croscarmellose sodium (cross-linked sodium carboxymethylcellulose) and LHPC (low-substituted hydroxypropylcellulose). The disintegrant or superdisintegrant may be present in an amount of from about 2% to about 90%, preferably from about 3% to 60% of the composition.

The ezetimibe product of these compositions and produced by the methods described herein may be administered to man or animal. The compositions described herein may be administered as pharmaceutical compositions. The ezetimibe composition may be administered in a therapeutically effective amount to a human or animal in need of such treatment. The term “therapeutically effective amount” as used herein refers to an amount of a pharmaceutical ingredient that is effective to treat, prevent or alleviate the symptoms of a disease. The pharmaceutical compositions of the present invention may be used alone or in combination with other anti-hyperlipidemia agents to treat a variety of diseases such as, but not limited to, the treatment of hyperlipidemia in children and adults.

Furthermore, compositions of the current invention may include additional active ingredients to the ezetimibe. Additional active pharmaceutical ingredients include, but are not limited to: analgesics, anti-arrhythmics, anti-bacterials, anti-convulsants, anti-Alzheimer's agents, anti-diabetics, anti-emetics, anti-fungals, anti-histiminics, anti-hyperlipidemics, anti-hyperlipoproteinemics, anti-hypertensives, anti-inflamatory agents, anti-Parkinsonian agents, anti-pulmonary hypertensives, anti-rheumatics, anti-ulceratives, anti-virals, cardiovascular agents, chemotherapy agents, central nervous system sedatives and stimulants, diuretics, gastrointestinal agents, hormones, respiratory agents, skin agents, as well as actives for the treatment of acne, benign prostatic hypertrophy, and irritable bowel syndrome.

The present invention is described in more detail by the following non-limiting examples.

### EXAMPLES

### Example 1

Two EZE spray dried products were produced from solutions containing concentration-enhancing polymers. One powder contained 1 EZE: 3 hydroxypropylmethylcellulose (HPMC) from 50% dichloromethane/50% methanol, while the other contained 1 EZE: 3 polyvinylpyrrolidone (PVP) from ethanol. The organic solvent solutions were spray dried using an SD-Micro® spray dryer (Niro, Inc.) to produce powder products.

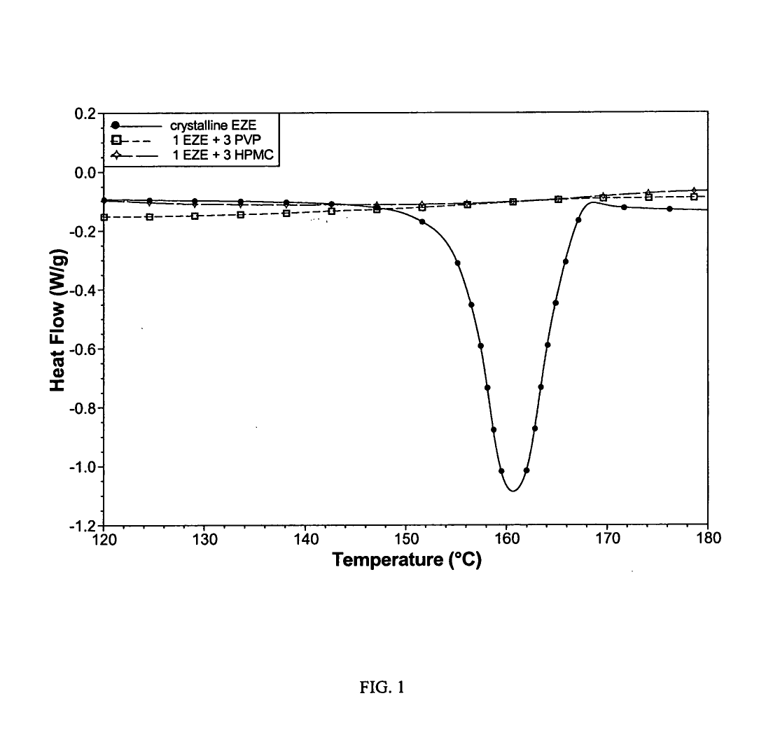

Both products contained only amorphous EZE, as revealed by DSC (FIG. 1).

### Example 2

The polyvinylpyrrolidone formula of Example 1 was modified by adding 2% (w/w) sodium lauryl sulfate (SLS) to the ethanol solution prior to spray drying to produce a powder.

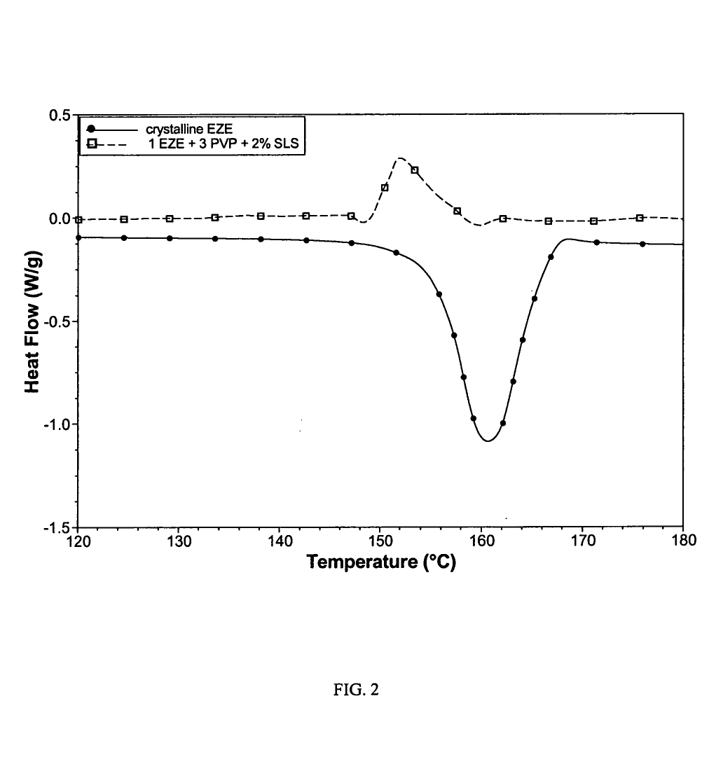

This product contained only amorphous EZE, as revealed by DSC (FIG. 2).

The dissolution properties of the spray dried powder were compared to the crystalline form of the drug. The spray dried product was hand-filled into hard gelatin capsules (Qualicaps®, Shinogi) with an additional 15% (w/w) croscarmellose sodium disintegrant. USP apparatus II (paddles) (VK 7010®, Varian Inc.) was used, with a bath temperature of 37° C. and a paddle speed of 100 rpm for 240 minutes. The dissolution medium was deaerated, filtered USP water.

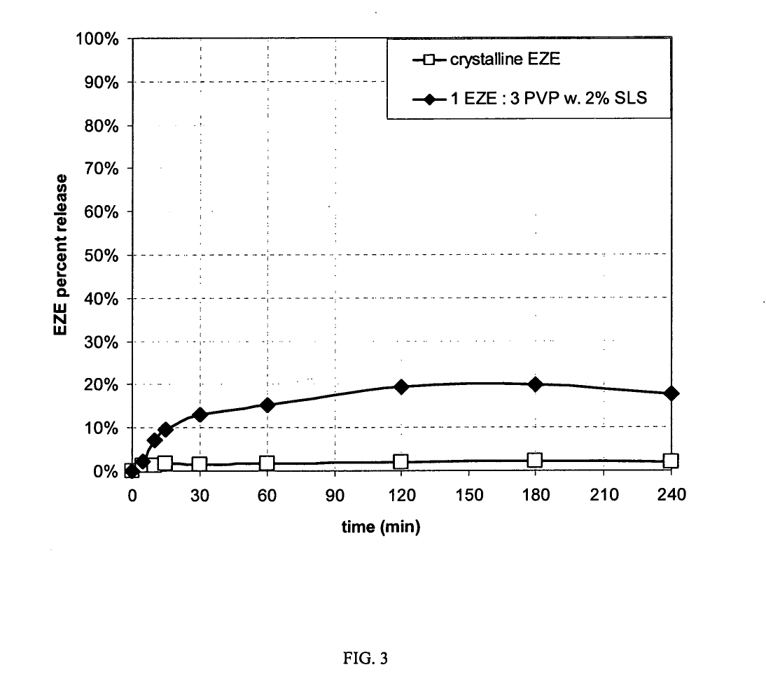

A ten-fold increase in EZE aqueous solubility was measured for the amorphous spray dried powder compared to the crystalline form (FIG. 3).

### Example 3

The pharmacokinetics of crystalline EZE and the amorphous 1 EZE: 3 PVP+2% SLS spray dried powder were studied in male Sprague Dawley rats in the fasted state. Six rats were single-dosed with either 100 mg/kg crystalline EZE or an equivalent amount of the amorphous EZE of Example 2.

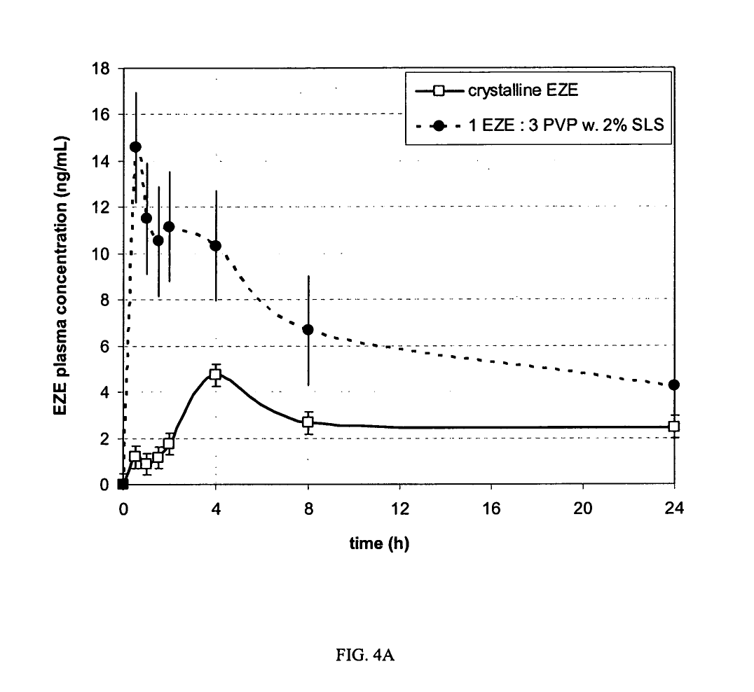

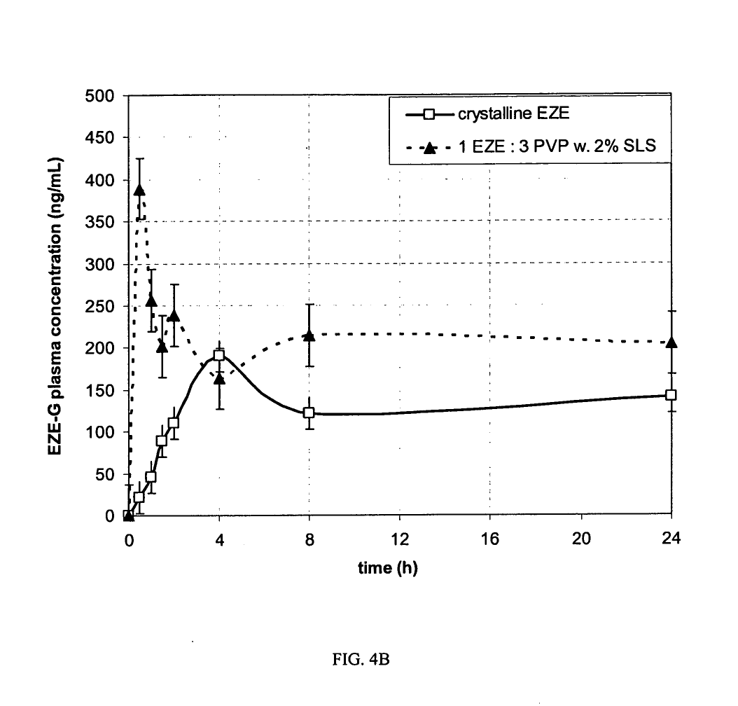

The amorphous EZE formulation enhanced the pharmacokinetics of ezetimibe and ezetimibe glucuronide (EZE-G), its active metabolite (Table 1, FIG. 4A-B). The amorphous composition of Example 2 increased the maximum plasma concentration (Cₘₐₓ) 3.7-fold for EZE and 1.9-fold for EZE-G, while decreasing the time to Cₘₐₓ by 5-7 hours. Drug exposure, as measured by the 0-24 hour area under the curve (AUC₀₋₂₄ₕ), increased 2.6-fold for EZE and 1.6-fold for EZE-G.

TABLE 1
Pharmacokinetic properties of crystalline vs. amorphous EZE.

| | | Cₘₐₓ | tₘₐₓ | AUC_{0–24 h} |
|---|---|---|---|---|
| molecule | drug form | (ng/mL) | (h) | (ng · h/mL) |
| EZE | crystalline | 5.1 | 8.0 | 64.2 |
| | amorphous | 19.1 | 0.8 | 164 |
| | composition | | | |
| EZE-G | crystalline | 245 | 6.0 | 3131 |
| | amorphous | 461 | 0.8 | 4990 |
| | composition | | | |

### Example 4

The bioenhanced polyvinylpyrrolidone formula of Example 3 was repeated by dissolving all components in a solvent blend of 80% ethanol/20% cyclohexane. Cyclohexane is a non-solvent for polyvinylpyrrolidone, as it does not expand the polymer molecule (α<1).

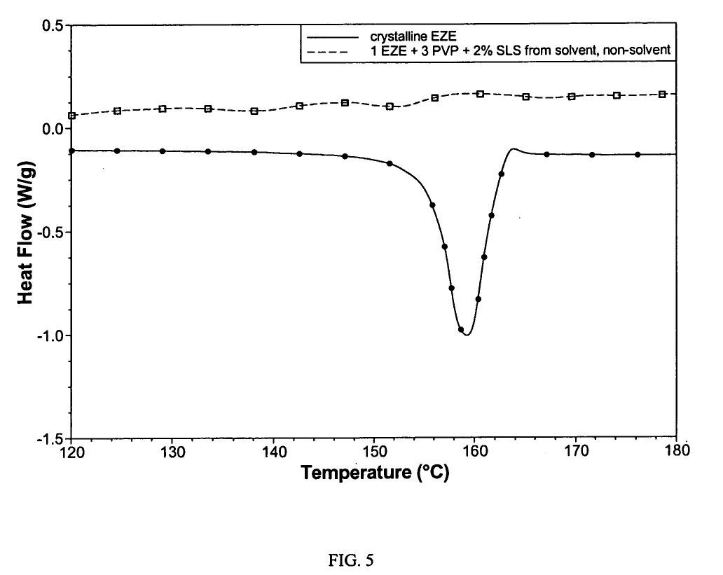

The spray dried product from solvent/non-solvent solution is amorphous, as detected by DSC (FIG. 5).

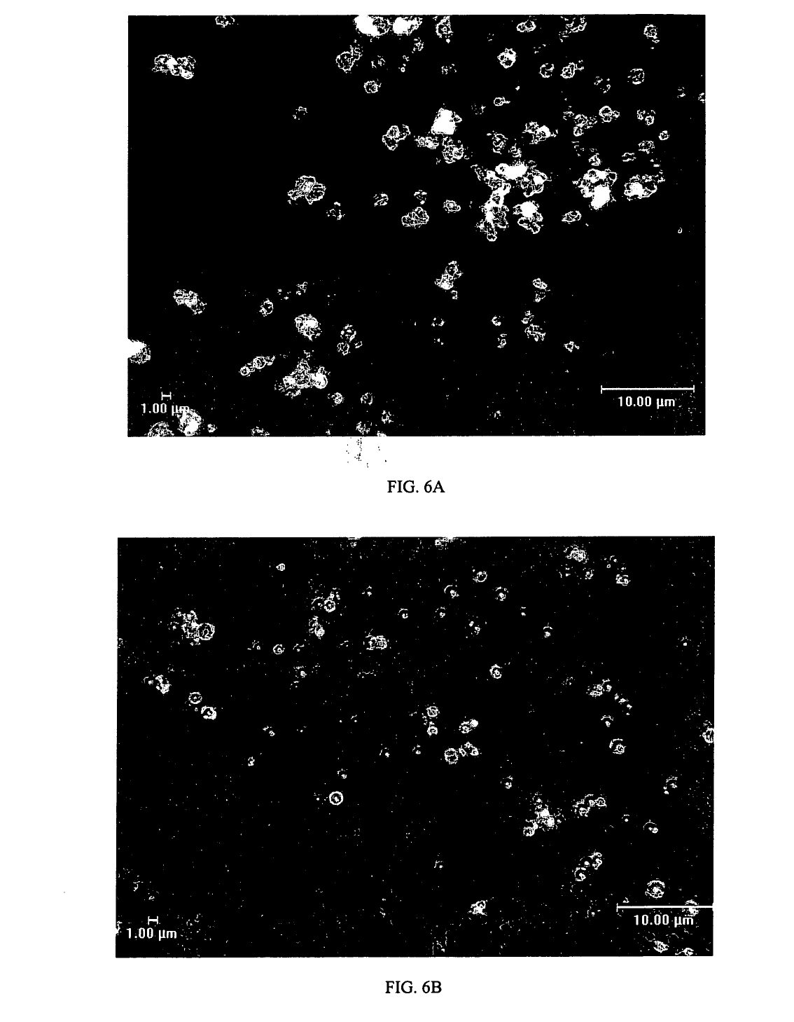

The addition of a polymer non-solvent altered the particle size distribution and morphology of the spray dried powder. Particles from solvent-only (Example 2) were not perfect, smooth spheres and instead possessed facets (FIG. 6A). As revealed by optical microscopy, the sizes of these particles were greater than about 1 μm. Spray dried particles from the solvent/non-solvent blend were spheres with a particle size less than about 1 μm (FIG. 6B).

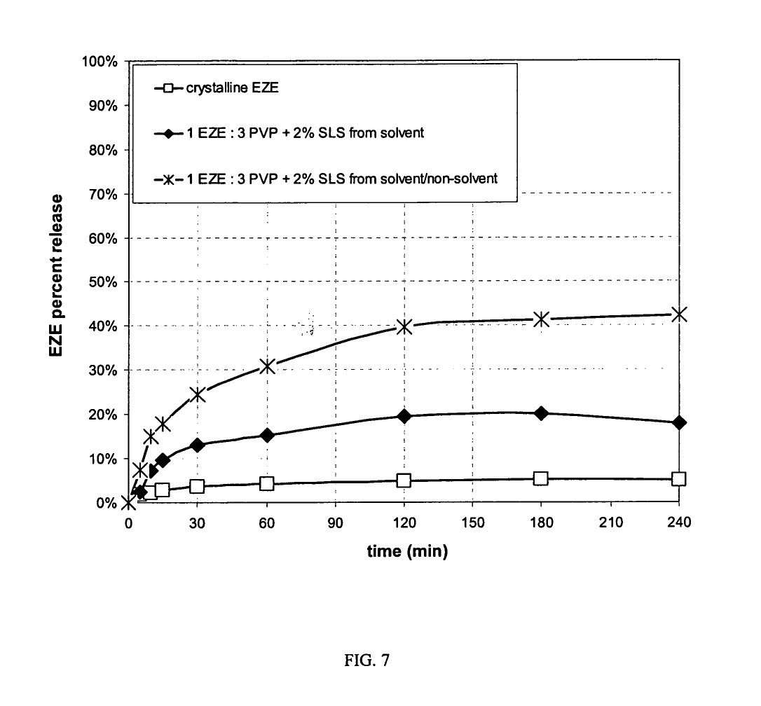

Adding a non-solvent to the working solution changed the release characteristics of the spray dried product. The dissolution sample preparation and experimental conditions were identical to those of Example 2. Both amorphous spray dried powders achieved faster and higher release than the crystalline form of EZE (FIG. 7). Yet, the initial rate of release (0-15 minutes) for the solvent/non-solvent powder was 80% higher than the solvent-only powder. The maximum release was higher for the solvent/non-solvent product (42% release) than the solvent product (18% release). Also, product from solvent-only solution appears to reach maximum concentration at 180 minutes, and then slightly decline. Spray dried product from solvent/non-solvent solution continues to release EZE during the entire test period.

Having described the invention in detail, it will be apparent that modifications and variations are possible without departing from the scope of the invention defined in the appended claims.

What is claimed is:

1. A composition comprising ezetimibe and a solubility-enhancing polymer wherein said ezetimibe exhibits enhanced bioavailability compared to a control composition without the solubility-enhancing polymer.

2. The composition of claim 1 wherein said ezetimibe is substantially amorphous.

3. The composition of claim 2 wherein said ezetimibe is almost completely amorphous.

4. The composition of claim 3 wherein said ezetimibe is completely amorphous.

5. The composition of claims 2, 3 or 4 wherein the ezetimibe and the solubility-enhancing polymer are present in the composition as a solid dispersion.

6. The composition of claim 1 wherein the polymer is selected from the group consisting of: aliphatic polyesters, carbohydrates, carboxyalkylcelluloses, alkylcelluloses, hydroxyalkylcelluloses, hydroxyalkylalkylcelluloses, hydroxyalkylalkylcellulose derivatives, polyamines, polyethylene glycols, methacrylic acid polymers and copolymers, homo- and copolymers of N-vinyl pyrrolidone, homo- and copolymers of vinyllactam, polysaccharides, poly glycols, polyvinyl esters, refined/modified shellac, and mixtures thereof.

7. The composition of claim 6 wherein the polymer comprises polyvinylpyrrolidone.

8. The composition of claim 1 wherein the ratio of ezetimibe to total polymer is between about 5% ezetimibe:95% total polymer to about 95% ezetimibe:5% total polymer.

9. The composition of claim 8 wherein the ratio of ezetimibe to solubility-enhancing polymer is between about 25% ezetimibe:75% polymer to about 75% ezetimibe:25% polymer.

10. The composition of claim 1 wherein the composition comprises spray dried particles of ezetimibe and polymer.

11. The composition of claim 10 wherein the spray dried particles of ezetimibe and polymer have an average particle size of from about 0.5 μm-500 μm

12. A pharmaceutical dosage form comprising the composition of claim 1.

13. The pharmaceutical dosage form of claim 12 wherein the dosage form comprises an oral, solid-dosage form.

14. The pharmaceutical dosage form of claim 13 wherein the dosage form comprises an oral, solid-dosage form selected from the group consisting of tablets, coated tablets, chewable tablets, capsules and gelatin capsules.

15. The pharmaceutical dosage form of claim 14 wherein the ezetimibe is substantially amorphous.

16. The pharmaceutical dosage form of claim 15 wherein the amorphous ezetimibe provides a maximum plasma concentration for a pharmaceutically active form of ezetimibe that is at least 1.25 times greater than that of a control composition containing crystalline ezetimibe.

17. The pharmaceutical dosage form of claim 16 wherein the amorphous ezetimibe provides a maximum plasma concentration for a pharmaceutically active form of ezetimibe that is at least 2 times greater than that of a control composition containing crystalline ezetimibe.

18. The pharmaceutical dosage form of claim 17 wherein the amorphous ezetimibe provides a maximum plasma concentration for a pharmaceutically active form of ezetimibe that is at least 3 times greater than that of a control composition containing crystalline ezetimibe.

19. The pharmaceutical dosage form of claim 15 wherein the amorphous ezetimibe provides an increase in the exposure (AUC₀₋₂₄ₕ) of at least 1.25 times that of a control composition containing crystalline ezetimibe.

20. The pharmaceutical dosage form of claim 19 wherein the amorphous ezetimibe provides an increase in the exposure (AUC₀₋₂₄ₕ) of at least 2 times that of a control composition containing crystalline ezetimibe.

21. The pharmaceutical dosage form of claim 20 wherein the amorphous ezetimibe provides an increase in the exposure (AUC₀₋₂₄ₕ) of at least 3 times that of a control composition containing crystalline ezetimibe.

22. The composition of claim 1 further comprising one or more ingredients selected from the group consisting of surfactant(s), pH modifier(s), filler(s), complexing agent(s), solubilizer(s), pigment(s), lubricant(s), glidant(s), flavor agent(s), plasticizer(s), taste masking agent(s), release-modifying polymer(s), and mixtures thereof.

23. A composition in accordance with claim 1 wherein the composition is in the form of a paste, solution, slurry, ointment, or dispersion.

24. The composition of claim 1 wherein at least one of the ezetimibe and the polymer is melt-processable.

25. A composition in accordance with claim 24 wherein the composition is in the form of an oral, solid-dosage form.

26. A composition in accordance with claim 25 wherein the oral, solid-dosage form comprises a tablet, a coated tablet, a chewable tablet, a capsule or a gelatin capsule.

27. A composition in accordance with claim 23 wherein the composition comprises pastes, solutions, slurries, ointments, or dispersions made from the ezetimibe-polymer melt product.

28. A method of preparing a composition comprising ezetimibe comprising:
contacting a quantity of ezetimibe with a solubility-enhancing polymer in a solvent for the polymer, thereby forming a mixture containing ezetimibe of enhanced bioavailability.

29. The method of claim 28 further comprising removing the solvent to form an ezetimibe-polymer composition.

30. The method of claim 28 wherein the ratio of ezetimibe to total polymer is between about 5% ezetimibe:95% total polymer to about 95% ezetimibe:5% total polymer.

31. The method of claim 28 wherein the composition further comprises one or more pharmaceutically acceptable ingredients.

32. The method of claim 28 wherein the polymer is selected from the group consisting of: aliphatic polyesters, carboxyalkylcelluloses, carbohydrates, alkylcelluloses, hydroxyalkylcelluloses, hydroxyalkylalkylcelluloses, hydroxyalkylalkylcellulose derivatives, polyamines, polyethylene glycols, methacrylic acid polymers and copolymers, homo- and copolymers of N-vinyl pyrrolidone, homo- and copolymers of vinyllactam, polysaccharides, poly glycols, polyvinyl esters, refined/modified shellac, and mixtures thereof.

33. The method of claim 32 wherein the polymer comprises polyvinylpyrrolidone.

34. The method of claim 28 wherein the mixture further comprises a non-solvent for the polymer.

35. The method of claim 34 wherein the solvent and non-solvent are present at a ratio of from about 5% solvent: 95% non-solvent to about 95% solvent: 5% non-solvent.

36. The method of claim 35 wherein the ratio of solvent to non-solvent is selected such that the polymer is dissolved in the solvent blend.

37. The method of claim 36 wherein the ezetimibe of enhanced bioavailability exhibits faster dissolution, greater extent of dissolution, or both compared to a ezetimibe composition made without a non-solvent for the polymer.

38. The method of claim 28 wherein the concentration of the polymer in the mixture is from about 1% to about 90%.

39. The method of claim 29 wherein the solvent is removed by spray drying the mixture to form particles comprising ezetimibe.

40. The method of claim 39 wherein said particles contain less than about 2% residual solvent.

41. The method of claim 28 wherein a major portion of said ezetimibe in said mixture is amorphous.

42. The method of claim 41 wherein the ezetimibe in said mixture is almost completely amorphous.

43. A composition comprising particles produced in accordance with claim 39.

44. An oral, solid-dosage form comprising particles produced in accordance with claim 39.

45. The oral, solid dosage form of claim 44 in the form of a capsule, a tablet, a chewable tablet, a granule, a bead, a gelatin capsule, or a pellet.

46. An ezetimibe product produced in accordance with claim 28.

47. A method for providing ezetimibe to a subject comprising administering to said subject the oral, solid dosage form of claim 45.

48. The method of claim 47 wherein said dosage form is administered to treat hyperlipidemia.

49. A composition comprising a solid dispersion of an ezetimibe and at least one solubility-enhancing polymer wherein said ezetimibe in said dispersion is substantially amorphous.

50. The composition of claim 49 wherein said ezetimibe in said dispersion is almost completely amorphous.

51. The method of claim 50 wherein the ezetimibe in said mixture is completely amorphous.

52. A method for preparing a composition comprising amorphous ezetimibe comprising:
a. providing a mixture comprising ezetimibe and solubility-enhancing polymer in a solvent or a blend of a solvent and non-solvent for the solubility-enhancing polymer;
b. distributing the mixture into either droplets or granules, and
c. evaporating the solvent or solvent and non-solvent from the mixture to form a composition comprising particles wherein the particles comprise amorphous ezetimibe.

53. The method of claim 52 wherein the particles have an average size of from about 0.5 μm to about 5000 μm.

54. The method of claim 52 wherein the mixture comprises a blend of a solvent and non-solvent for the solubility-enhancing polymer.

55. The method of claim 54 wherein said particles possess less crystalline drug than particles produced from a mixture containing solvent alone.

56. The method of claim 53 wherein the mixture comprises a solubility-enhancing polymer/solvent/non-solvent combination selected from the group consisting of polyvinylpyrrolidone/dichloromethane/acetone, polyvinylpyrrolidone-co-vinyl acetate/acetone/hexane, and ethylcellulose/acetone/water.

57. A composition comprising ezetimibe and a solubility-enhancing polymer wherein said composition exhibits at least one of the following compared to a control composition without the solubility-enhancing polymer:
a) an increase in initial release of at least about 25%
b) an increase in extent of release of at least about 25%
c) an increase in maximum plasma concentration of at least about 25%
d) an increase in AUC₀₋₈ₕ of at least about 25%.

58. The composition of claim 57 wherein the solubility-enhancing polymer is selected from the group consisting of aliphatic polyesters, carboxyalkyl celluloses, alkylcelluloses, hydroxyalkyl celluloses, hydroxyalkylalkyl celluloses, hydroxyalkylalkyl cellulose derivatives, polyamines, polyethylene glycols, methacrylic acid polymers and copolymers, homo- and copolymers of N-vinyl pyrrolidone, homo- and copolymers of vinyllactam, polysaccharides, poly glycols, polyvinyl esters, refined/modified shellac and mixtures thereof.

59. The composition of claim 57 wherein said composition comprises spray dried particles of ezetimibe and solubility-enhancing polymer.

60. The composition of claim 57 wherein the ezetimibe is substantially amorphous.

61. The composition of claim 60 wherein said ezetimibe in said composition is almost completely amorphous.

62. The method of claim 61 wherein the ezetimibe in said composition is completely amorphous.
